(19) United States
(12) Patent Application Publication
(10) Pub. No.: US 2003/0202902 A1
(43) Pub. Date: Oct. 30, 2003

(54) Dishcloth sanitizing frame

(76) Inventor: James B. Elliott, Hacienda Heights, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/423,510

(22) Filed: Apr. 24, 2003

Related U.S. Application Data
(60) Provisional application No. 60/375,035, filed on Apr. 24, 2002.

(51) Int. Cl. A61L 2/18; A61L 2/00

(52) U.S. Cl. 422/28; 422/1; 422/292; 422/300; 422/301

(57) ABSTRACT

A dishcloth sanitizing frame is provided. The sanitizing frame comprises first and second panels, each panel comprising a generally open framework and inner and outer surfaces. The panels can be removably joined to each other so that, when the frame is in a closed position, a dishcloth can be held between and in contact with the inner surfaces of the panels. A dishcloth can be sanitized by introducing the dishcloth into the sanitizing frame between the inner surfaces of the panels. The dishcloth sanitizing frame and dishcloth are placed into an automatic dishwasher, and the dishcloth is washed, and optionally dried, in the automatic dishwasher.

### CROSS-REFERENCE TO RELATED APPLICATION(S)

This application claims priority to U.S. Provisional Application No. 60/375,035, filed Apr. 24, 2002, the entire disclosure of which is incorporated herein.

### BACKGROUND

The common dishcloth has been identified as the most biologically contaminated item in the typical kitchen of even the most fastidious housekeepers. In fact, the dishcloth is much more contaminated with bacteria in most households than water in the toilet bowl. No one knows how many intestestinal disorders and other maladies result from this common source of bacterial contamination. Nonetheless, it is clear that the high levels of contamination commonly carried on a dishcloth pose an invisible but real threat to family health.

The foods we prepare and consume in our homes are also food for many types of bacteria. These bacteria are present in our environment all the time. But even though they may be potentially dangerous, in the moderate numbers normally encountered, our body's defenses are usually able to protect us. However if these common bacteria are allowed to get into our body in large enough numbers, by contamination of our food or transfer from our contaminated hands, they can overpower our body's defenses and make us sick. They can also lower our immunity to other opportunistic infections, and many have the potential to even kill us.

Bacteria require three conditions to grow or incubate. The first is contact with food—most any organic material will do, but especially the kinds of things we eat. The second is water or any source of moisture. In dry conditions they become dormant (referred to as spores) and wait for the return of moisture. The third is moderate temperature, generally from 40 to 120° F., with the ideal temperature range being from 70 to 90° F., kind of like our preference in our homes. Below 40° they become inactive and above 120° they start to die.

When bacteria have a source of moist food in a room temperature environment they grow rapidly and multiply exponentially by the millions or billions.

The common dishcloth has several typical uses. It is used to wipe food remnants from the dinner dishes, food preparation utensils, and table/counter surfaces (all laden with bacteria), thereby filling the porous cloth material with a mixture of food particles and bacteria. The dishcloth is often then given a quick rinse and a squeeze to remove most of the visible food pieces, but leaving it full of microscopic food particles, bacteria and moisture. Then the dishcloth is draped over the edge of the sink or faucet, or perhaps hang it in the cabinet under the sink (where a garbage container is often kept, so that a high concentration of bacteria is floating in the air). Too often it is just left sopping in the corner of the sink. By this typical “cleanup” process, a perfect bacterial incubator has just been set up, where the bacteria will thrive and multiply all night (or at least for a few hours until the dishcloth dries, depending on how and where it is left). Then the next day the process is repeated, not just once, but two, three or more times. Meanwhile, the increasingly contaminated dishcloth is used to spread millions of bacteria on hands, dishes and food preparation surfaces.

Professional cooks in commercial kitchens normally address the issue of bacterial build up in their cleaning and handling cloths by discarding them at least once each meal and replacing with freshly laundered and disinfected cloths (usually required by local health laws). However, not many homemakers are willing to deal with a set of 21 or more dishcloths (3 meals, 7 days), or alternatively to launder a smaller number several times a week, just to maintain a bacteria-safe dishcloth.

Many persons use sponges instead of a standard dishcloth, thinking that they are somehow safer than a dishcloth. After all, many of them claim to be anti-bacterial. The fine print on the package typically indicates that the product is bacteriostatic or bio-static, meaning that the item is made of materials that will not support bacterial/microbial growth. In other words, if you keep the sponge absolutely clean (do not add any organic material), the sponge material will not allow bacteria to thrive and multiply by consuming the sponge. This does not mean, however, that if it is contaminated with organic material (unavoidable if it is used for its intended purpose) the sponge will prevent bacteria from eating and thriving on that food. In fact, sponges due to their thickness and micro-porous form tend to be actually more efficient bacterial incubators than many dishcloths. This is because they tend to retain more microscopic food particles and they dry slower. If the manufacturers were to produce a truly bactericidal sponge, it would almost certainly violate regulatory standards for chemical or radiological safety and would probably be dangerous to contact food preparation or serving utensils. The above limited-value bacteriostatic “anti-bacterial” action is probably short lived and potentially exceeded by the sponge's retention time in a typical household. It should also be noted that any bacterium genetically predisposed to eat the materials in a sponge is probably harmless to humans anyway.

Another method recommended by some “authorities” and apparently used by some people is to “nuke” the dishcloth in a microwave oven for several seconds. The apparent theory is that the microwave energy will raise the temperature of the dishcloth above 140° F. long enough to kill the bacteria present. However, a minimal pasteurization cycle to kill most of the harmful bacteria in foodstuffs is 140° F. for 20 minutes, and full sterilization requires much more temperature/time, such as boiling in water for 30 minutes. One can estimate the relative effectiveness (ineffectiveness) of this method of bacterial control, allowing generously conservative values in the assumptions for the key parameters of our hypothetical method. For the purposes of this exercise, we can assume that users always perform this hypothetical microwave process under ideal conditions and in a perfectly repeatable way once each day, and that one can thereby achieve a 90% kill (10% survival) rate of all harmful bacteria. For the microwave method to work, the dishcloth by definition has to be wet or moist (the microwaves used only act on water molecules). This means that although we have killed some of the bacteria, those that are left (10%) still have everything they need to survive and thrive: food, moisture and warmth. So, in the interim between treatments, the bacteria are again busily multiplying exponentially.

To simplify the calculations, we can conservatively assume the bacteria are at least doubling in numbers every 3 hours (actually much more rapidly under typical conditions). So in 3 hours we will have 20% of the bacteria we started with, in 6 hours 40%, in 9 hours 80%, in 12 hours 160%, in 15 hours 320%, in 18 hours 640%, in 21 hours 1,280%. Therefore in 24 hours when we repeat the microwave treatment process we will have 2,560% of the original contamination . . . over 25 times the number of bacteria we started with. If we again perform the hypothetical process perfectly, we will again kill 90% of the bacteria present, but that means that 10% of the 2,560% or 256% of the original contamination is now left. In 3 hours this will become 512%, 6 hours 1,024%, etc. It is not difficult to see where this is going.

Knowing human nature and the pace of life in the typical household, one can assume that most persons who use this method probably do so only occasionally, rather than as a consistent routine. If the time period between microwave treatments were 36 hours (just a half day longer) for example, using our same conservative assumptions for analysis, the level of contamination would grow to not just 25 times, but 400 times the original bacteria count. So, even if this microwave method could guaranty a 98 or 99% kill rate, the predictable real-life net effect is that most users would still have a highly contaminated dishcloth in only a few days of use.

It should be pointed out that in the above simplified analysis we have not taken into account the added bacterial contamination resulting from the other meals/uses of the day, just the survival and growth of the original bacterial contamination from one meal. We have meanwhile been transferring high levels of bacteria onto our dishes and other serving utensils, food preparation surfaces, and our hands. The fact should be noted that this microwave process is itself potentially dangerous. A simple mistake in the microwave power level/time settings can cause scorching damage to the dishcloth, and may start a fire in the microwave oven.

Mitigating any of the three basic bacterial incubation factors can slow bacterial growth, but if all three factors can be addressed in one sanitizing method, synergy can be achieved and truly significant results are possible. Any sanitizing method, however powerful, needs to be consistently applied by the user to be effective. To become consistently used, the method must be simple, convenient and safe for the typical user to adopt and habitually follow as part of their normal routine for after-meal kitchen cleanup.

### SUMMARY OF THE INVENTION

The present invention is directed to significantly reducing or even eliminating all three bacterial incubation factors elements, increasing the useful life of a dishcloth. In one embodiment, the invention is directed to a dishcloth sanitizing frame comprising first and second panels, each panel comprising a generally open framework and having inner and outer surfaces, wherein the panels can be removably joined to each other so that, when the frame is in a closed position, a dishcloth can be held between and in contact with the inner surfaces of the panels.

The invention is also directed to a method for sanitizing a dishcloth. The method comprises introducing a dishcloth between the inner surfaces of the panels of a dishcloth sanitizing frame as described above. The dishcloth sanitizing frame and dishcloth are placed into a dishwasher, and the dishcloth is washed, and optionally dried, in the dishwasher. As used herein, the term “washed” does not require the presence of a detergent, but includes washing with only water.

### BRIEF DESCRIPTION OF THE DRAWINGS

FIG. 1A is a perspective view of a dishcloth sanitizing frame according to the invention in a closed position and containing a dishcloth.

FIG. 1B is a perspective view of the dishcloth sanitizing frame of FIG. 1A in an open position.

### DETAILED DESCRIPTION OF THE INVENTION

The present invention is directed to a dishcloth sanitizing frame and a method for its use. This inventive dishcloth sanitizing frame allows a dishcloth to be securely captured and held, preferably without folds, gathers or pleats, and sent through an automatic dishwasher wash/dry cycle with dishes, pots, and pans. This can be done with each washer load and will reduce the bacterial contamination to far safer levels, particularly with the new anti-bacterial washing detergents and heated drying cycle.

As used herein, the term “dishcloth” should be understood to apply equally to any fabric, sponge or similar material implement commonly used for hand-washing of dishes, utensils or other food contacting items, as well as general cleanup of kitchen food preparation surfaces.

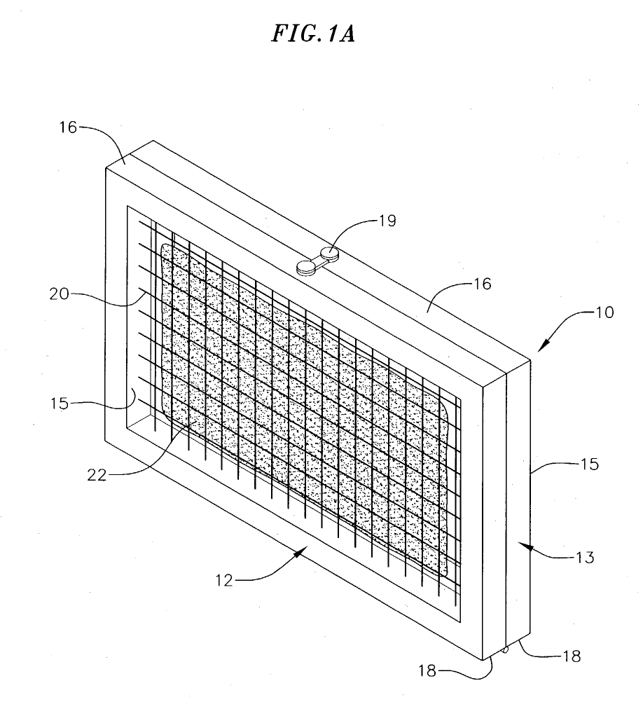

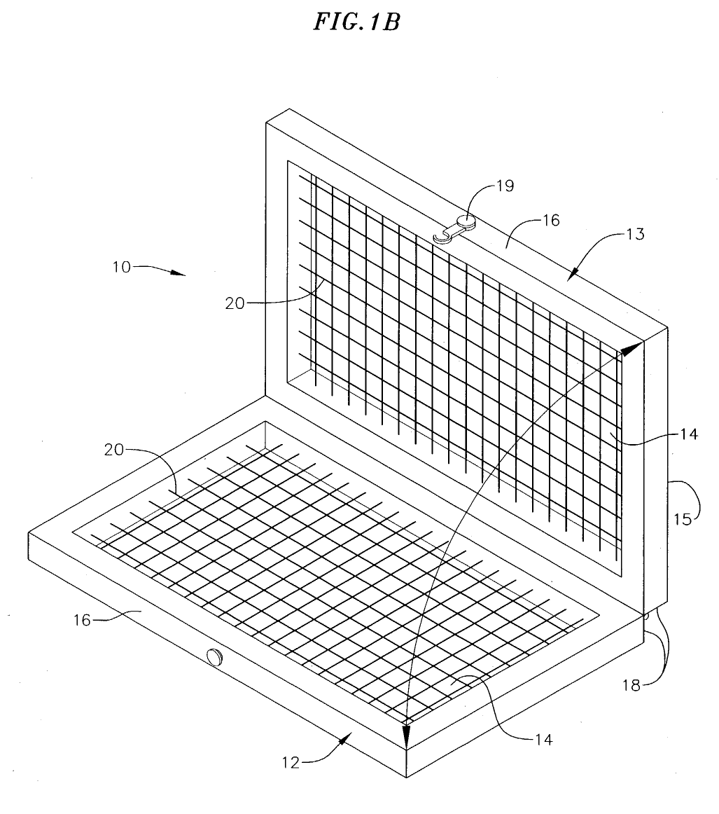

In one embodiment, the dishcloth sanitizing frame is generally book-shaped, as shown in FIGS. 1A and 1B. The frame 10 comprises a first panel 12 and a second panel 13, each having an inner surface 14, an outer surface 15 and opposing top and bottom edges 16 and 18. The first and second panels 12 and 13 are joined to each other at their opposing top and bottom edges 16 and 18 so that their inner surfaces 14 face generally toward each other and their outer surfaces 15 face generally away from each other. In the depicted embodiment, the inner surfaces 14 and outer surfaces 15 are all generally parallel to each other, although other arrangements are contemplated within the scope of the invention.

Each panel 12 and 13 is preferably generally rectangular, but other shapes can also be used, such as circular or oval. One would recognize that, for example, in the case of a circle, where the frame has a single continuous edge, two regions of the edge, preferably two opposing regions, could be joined to each other. Accordingly, the invention does not require the joining of two opposite edges.

The depicted dishcloth sanitizing frame comprises rectangular first and second panels 12 and 13 that have are permanently hingedly-attached at their bottom edges 16. The panels are removably joined at their top edges with a latch 19 that, in use, keeps the panels in a securely closed position, as shown in FIG. 1A. The invention includes other arrangements and mechanisms for joining the panels 12 and 13 so that at least one of their edges, such as the top edges 16, are removably joined to each other, including arrangements where other edges, such as the bottom edges 18, are removably joined to each other and where the bottom edges 18 are permanently joined to each other.

Each panel 12 and 13 comprises a generally open framework between the top 16 and bottom edges 18. The depicted embodiment includes a support grid 20 or screen that is mostly open, so that a dishcloth placed inside the sanitizing frame can be supported in a flat plane and exposed on both sides to the washer spray action during the wash cycle. Frameworks having openings of other sizes and shapes, such a triangular or hexagonal, are contemplated within the scope of the invention, and can depend on the desired aesthetic and/or functional characteristics of the frame. Preferably each panel is open over at least about 60%, 70%, 80% or 90% of its surface area, although this can similarly vary depending on the desired aesthetic and/or functional characteristics of the frame. The frame need only contact the dishcloth as required to support and maintain the dishcloth in position for all spatial orientations of the frame.

The height, width, and thickness of the sanitizing frame are such that, with a dishcloth 22 inside, as shown in FIG. 1A, it will fit in one of the sections of a dishwasher loading rack where dinner plates are normally placed. In a preferred embodiment, the sanitizing frame has a height and width ranging from about 4 to about 20 inches, more preferably from about 8 to about 14 inches and a thickness ranging from about 0.25 to about 1.5 inches, more preferably from about 0.25 to about 1 inch.

In a preferred embodiment, the panels 12 and 13 are generally identical in design, with integral attachment features, to reduce design complexity and manufacturing cost.

The sanitizing frame is made of one or more material that will not corrode or be otherwise damaged by repeated cycles in a home dishwasher. Examples of suitable materials include stainless and chrome-plated steel, high-density polyethylene, nylon, polypropylene, and various copolymer alloys and blends, such as ABS (acrylic-butylene-styrene). Notably, the polymer materials can be produced with precision in high volumes and at low cost by standard injection molding processes.

The design of the invention can be easily made to accommodate various thickness dishcloths by a variety of methods. In one embodiment, when the sanitizing frame is in the closed position, a space is provided between the inner surfaces 14 of the panels 12 and 13. The space can be any suitable size, for example, from about 1/64 inch to about ¼ inch. Alternatively, the frame can include flexible features for joining the panels 12 and 13.

If desired, finger-like extensions perpendicular to the principal plane of the support grid, or similar features, could be provided to further enhance retention of the dishcloth. An array of finger or hook features attached to the framework could also be employed to enhance or replace the support grid. Such finger or hook features could be employed and arranged in an embodiment of the invention so as to require only a single framework panel.

Typical use of the inventive sanitizing frame will occur after loading of the dishes and utensils from a meal into the dishwasher and other uses of the dishcloth are finished. With reference to the above-described sanitizing frame, the dishcloth is then spread on the inner surface 14 of one of the panels 12 or 13 or otherwise situated generally between the inner surfaces of the panels. The panels 12 and 13 are moved toward each other to thereby enclose the dishcloth and close the sanitizing frame. The top edges 16 of the panels 12 and 13 are latched, trapping the dishcloth between the panels. The sanitizing frame is then placed in the dishwasher, such as an automatic or industrial dishwasher, with or without the dishes, and the wash cycle started.

During the wash cycle, the dishcloth is impinged on both sides by a prolonged forceful spray of detergent solution and then clean rinse water along with the other contents of the machine. This results in a much more thorough removal of contaminating microbes, food particles and other debris than the typical quick hand rinse and squeeze that most dishcloths get. Alternatively, the dishcloth in the sanitizing frame could be exposed to a rinse cycle in the dishwasher without the use of detergent, which can still provide beneficial results.

The fact that the dishwasher next goes immediately into a drying cycle further enhances the efficiency of the sanitizing action. As noted previously, bacteria need moisture to thrive and multiply. When dry, they enter a dormant state. So, after the dishcloth is washed in the dishwasher in accordance with the invention, the relatively few microbes remaining are now stopped from multiplying by the drying cycle. If the an anti-bacterial washing detergent and/or an elevated-temperature “sanitizing” cycle is used, the results are further improved. If an elevated-temperature drying cycle is not used, it may be desirable to remove the sanitizing frame and dishcloth from the dishwasher after the completion of the wash cycle. The dishcloth could then be hung to dry outside the dishwasher where it could dry faster, thereby preventing growth of any surviving bacteria.

Loading the dishcloth in the sanitizing frame may be somewhat different for other embodiments of the invention, but the sanitizing process is generally the same.

Routine use of this invention as described above reduces microbial contamination of the typical dishcloth by several orders of magnitude. Reduction of at least 100 to one can be realistically expected, even without anti-bacterial detergents. The thorough washing and rinsing of the dishcloth by the dishwasher addresses the first two of the putrefaction causes noted above, microbial contamination and nutrients. The forced drying of the dishcloth addresses the third, moisture.

What is claimed is:

1. A dishcloth sanitizing frame comprising first and second panels, each panel comprising a generally open framework and having inner and outer surfaces, wherein the panels can be removably joined to each other so that, when the frame is in a closed position, a dishcloth can be held between and in contact with the inner surfaces of the panels.

2. The dishcloth sanitizing frame according to claim 1, wherein the frame has a thickness ranging from about 0.25 to about 1.5 inches.

3. The dishcloth sanitizing frame according to claim 1, wherein the frame has a thickness ranging from about 0.25 to about 1 inch.

4. The dishcloth sanitizing frame according to claim 1, wherein the frame has a height ranging from about 4 to about 20 inches and a width ranging from about 4 to about 20 inches.

5. The dishcloth sanitizing frame according to claim 1, wherein the frame has a height ranging from about 8 to about 14 inches and a width ranging from about 8 to about 14 inches.

6. The dishcloth sanitizing frame according to claim 5, wherein the frame has a thickness ranging from about 0.25 to about 1 inch.

7. The dishcloth sanitizing frame according to claim 1, wherein the first and second panels are each generally rectangular and have top and bottom edges.

8. The dishcloth sanitizing frame according to claim 7, wherein the top edges of the panels are removably joined to each other.

9. The dishcloth sanitizing frame according to claim 8, wherein the top edges of the panels are removably joined to each other with a latch.

10. The dishcloth sanitizing frame according to claim 7, wherein the bottom edges of the panels are removably joined to each other.

11. The dishcloth sanitizing frame according to claim 7, wherein the bottom edges of the panels are permanently joined to each other.

12. The dishcloth sanitizing frame according to claim 7, wherein the bottom edges of the panels are hingedly joined to each other.

13. The dishcloth sanitizing frame according to claim 1, wherein each panel is open over at least about 60% of its surface area.

14. The dishcloth sanitizing frame according to claim 1, wherein each panel is open over at least about 70% of its surface area.

15. The dishcloth sanitizing frame according to claim 1, wherein each panel is open over at least about 80% of its surface area.

16. The dishcloth sanitizing frame according to claim 1, wherein each panel is open over at least about 90% of its surface area.

17. A method for sanitizing a dishcloth comprising:
introducing a dishcloth between the inner surfaces of the panels of the dishcloth sanitizing frame according to claim 1;
placing the dishcloth sanitizing frame and dishcloth into a dishwasher; and
washing the dishcloth in the dishwasher.

18. The method according to claim 17, wherein the dishwasher is an automatic dishwasher.

19. The method according to claim 17, wherein the dishcloth is washed in the dishwasher using a detergent

20. The method according to claim 17, wherein the dishcloth is washed in the dishwasher using an anti-bacterial detergent.

21. The method according to claim 17, further comprising drying the dishcloth in the automatic dishwasher using an elevated-temperature drying cycle.

22. The method according to claim 17, further comprising removing the washed dishcloth while it is wet and thereafter drying the dishcloth outside the dishwasher.
